(19) United States
(12) Patent Application Publication
(10) Pub. No.: US 2024/0062131 A1
(43) Pub. Date: Feb. 22, 2024

(54) Data Driven Multi-Criteria Optimization of Chemical Formulations

(71) Applicants: BASF SE, Ludwigshafen am Rhein (DE); Fraunhofer-Gesellschaft Zur Forderung Der Angewandten Forschung E.V., Munchen (DE)

(72) Inventors: David Hajnal, Ludwigshafen (DE); Stefan Lehner, Ludwigshafen (DE); Julia Reske, Ludwigshafen (DE); Michael Bortz, Kaiserslautern (DE); Karl-Heinz Kuefer, Kaiserslautern (DE); Philipp Suess, Kaiserslautern (DE)

(21) Appl. No.: 18/268,986

(22) PCT Filed: Dec. 17, 2021
(86) PCT No.: PCT/EP2021/086462

§ 371 Date: Jun. 22, 2023

(30) Foreign Application Priority Data
Dec. 23, 2020 (EP) 20216852.2

(51) Int. Cl. G06Q 10/0633; G06Q 10/0639; G06Q 50/04

(52) U.S. Cl. CPC G06Q 10/0633; G06Q 10/06395; G06Q 50/04

(57) ABSTRACT

The present invention relates generally to the field of chemical formulations in a chemical production facility, and more particularly to providing assistance for producing a chemical formulation in a chemical production facility. In detail, the present invention relates to a computer-implemented method for providing assistance for optimizing chemical formulations, comprising: (a) receiving input data, preferably via an input unit, of at least one set of experimental data comprising formulation data and/or process data, key physicochemical properties of the formulation and a target product profile, TPP, comprising a minimum product requirement, (b) performing multicriterial optimization based on a computational model based on experimental data via a processing unit and (c) providing optimization signal, preferably via an output unit, wherein the optimization signal is configured to control and/or monitor, preferably via a control unit, the production process of the chemical formulation

The present invention relates generally to the field of chemical formulations, and more particularly to providing assistance for producing a chemical formulation in a chemical production facility. In detail, the present invention relates to a computer-implemented method for producing a chemical formulation in a chemical production facility, preferably for guiding the production of a chemical formulation in a chemical production facility, comprising: (a) receiving input data, preferably via an input unit (10), of at least one set of experimental data comprising formulation data and/or process data and/or key physicochemical properties of the ingredients and/or the formulation and a target product profile, TPP, comprising a minimum product requirement, (b) performing multicriterial optimization based on a computational model based on experimental data via a processing unit (20) and (c) providing optimization signal, preferably via an output unit (30), wherein the optimization signal is configured to control and/or monitor, preferably via a control unit (40), the production process of the chemical formulation.

The present invention also relates to an apparatus for producing a chemical formulation in a chemical production facility, preferably for guiding the production of a chemical formulation in a chemical production facility, comprising: an input unit (10); an output unit (30) and
a processing unit (20) configured to: a) receive a user input, via the input unit, wherein the user input defines: at least one set of experimental data comprising formulation data and/or process data and/or key physicochemical properties of the ingredients and/or the formulation and a target product profile, TPP, comprising a minimum product requirement b) perform, via the processing unit (20), multicriterial optimization based on a computational model using experimental data c) provide, via the output unit, optimization results;
and to a system comprising said apparatus. The present invention further relates to methods, computer programs, data carriers, and uses related to the aforesaid method, apparatus, and system.

Processes for production of complex compositions such as those found in many chemical products generally require the mixing of many ingredients according to specific process parameters regarding formulation and production technology, to provide the product with properties at a level offering satisfactory performance according to predetermined specifications. In such complex production processes, it is not unusual that some process parameters involved exhibit interfering effects on the desired properties, further complicating the process design. Where possible, the designer may try to adapt the set of process parameters from known data derived from previous similar processes, and/or rely on conventional trial-and-error experimental schemes to optimize the set of process parameters values, in order to meet the product specifications. However, as the processes become more complex, optimization in such multidimensional space with high accuracy requirements turns out to be an extremely difficult task, even for the highly skilled designer. That limitation is particularly problematic in the design of chemical products, where one or more main ingredients mixed with a variety of auxiliaries and additives must be produced in the form of a stable and processible suspension, dispersion or solution.

In practical process and formulation design, the engineer often tries to find an optimal solution in the multi-dimensional objective space by an empirical iterative change of the process and formulation parameters in the design space. Usually, this procedure is continued until either a solution is found which fulfills certain requirements, or some deadline is reached where a solution has to be delivered. This procedure may lead to good results, however, no guarantee on optimality can be given. Furthermore, the empirical optimization only covers restricted areas in both the design and objective space, so that only limited information on the trade-offs between the different objectives is available and the decision cannot be based on an overview of the full solution space. This limitation may lead to overlooking interesting solutions. It is by far not necessary to calculate all feasible solutions. In principle, only best compromises need to be studied. These are solutions where an improvement in any objective can only be achieved by accepting a worsening in at least one other objective. The respective solutions are called Pareto-optimal (Geoffrion, 1968) and the set comprising all these solutions is the Pareto set, often also called the Pareto frontier because the Pareto set lies on the border between feasible and infeasible solutions. A common strategy to find single Pareto-optimal solutions is to weight the objectives and subsequently optimize the weighted sum. A drawback of this approach is that the weights have to be chosen beforehand. As the choice of the weights is ambiguous, many solutions will not be accepted without exploring alternative choices. Hence, this approach is, in practice, also empirical and iterative. Furthermore, it is difficult to extend this approach to “soft” objectives, describing safety, environmental, sustainable, or social issues. These are difficult to weight and compare directly to technical or economic objectives. Moreover, not every Pareto-optimal solution can be found by the weighted sum approach: namely if the Pareto set is not convex (Chankong & Haimes, 1983; Haimes, 1977).

There is, thus, a need in the art to provide reliable means and methods for multicriteria optimization of chemical formulations. In particular, there is a need to provide means and methods avoiding at least in part the drawbacks of the prior art as discussed above.

This problem is solved by the methods, apparatus, system, and uses with the features of the independent claims. Preferred embodiments, which might be realized in an isolated fashion or in any arbitrary combination are listed in the dependent claims.

This may help the users (e.g. a business) to identify and produce suitable formulations. The number of lab experiments would be reduced to an absolute minimum. This would speed up formulation development and production and save cost.

Accordingly, the present invention relates to a computer-implemented method for producing a chemical formulation in a chemical production facility, preferably for guiding the production of a chemical formulation in a chemical production facility, comprising:
(a) receiving input data, preferably via an input unit (10), of at least one set of experimental data comprising formulation data and/or process data and/or key physicochemical properties of the ingredients and/or the formulation and a target product profile, TPP, comprising a minimum product requirement,
(b) performing multicriterial optimization based on a computational model based on experimental data via a processing unit (20);
(c) providing optimization signal, preferably via an output unit (30), wherein the optimization signal is configured to control and/or monitor, preferably via a control unit (40), the production process of the chemical formulation.

The method of the present invention may comprise steps in addition to those explicitly mentioned above. For example, further steps may relate, e.g., to specific steps of optimizing the chemical formulation or combinations of such steps, preferably as indicated herein in the claims and/or the embodiments. Preferably, the method additionally comprises data filtering, quality checks the data, detecting outliers, estimating and/or augmenting missing values, normalizing data, merging data, data imputation, data reduction, applying dimension reduction techniques, selecting subsets of data and/or sorting data. Preferably, the method is used in any process for producing a chemical formulation, e.g. in coatings and paints, adhesives, in the field of crop protection and fertilization, in seed treatment, in laundry processes (e.g. in a washing machine, a dishwasher, or an industrial laundry machine), in food (e.g. milk, or meat) processing, in animal feed processing, in biofuel production, in leather production, in textile production, in pulp and paper industry, in beverage production, in chemical production processes, in water treatment, and/or in the field of human and veterinary medicine etc.

Moreover, the method may be preceded by steps establishing a computational model of chemical formulations, e.g. models which are capable to predict the target application profile or any element thereof from the formulation data and/or process data and/or key physicochemical properties of the ingredients and/or the formulation or from any possible selected subset thereof. More specifically, the models are mathematical functions whose independent (input) variables are the formulation data and/or process data and/or key physicochemical properties of the ingredients and/or the formulation or any possible selected subset thereof, and whose dependent (output) variables are defined by the parameters forming the target application profile ore any part/subset thereof. The mathematical functions can be based on statistical/empirical ansatz function calibrated with the data specified above, or can be based on tabulated data, or may be consisting of empirical/semi-empirical/first principle equations from general science like but nut limited to physics, chemistry, engineering, mathematics, statistics, economics. The mathematical functions may also consist of all possible combinations and or subsets of the above mentioned examples. More explicitly, regression models/functions linking the input variables to the target application profile can be used. Preferentially, these are neural network models, support vector machines, kernel based regressions, Gaussian process models, tree based models, random forest models, locally adaptive models, polynomial models, models including dimension reduction steps like not limited to principal component regression, partial least squares models, auto encoder. Preferentially, polynomial models of arbitrary order are used. More preferentially, second order polynomial models are used, consisting of quadratic terms, interaction terms, linear terms, and constant terms (model intercept terms). It is also preferred to use linear models. More preferentially linear models including a dimension reduction algorithm like partial least squares models are used. It is of course also possible to use combinations of the above mentioned models.

Referring to the computer-implemented aspects of the invention, one or more of the method steps, preferably all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual key physicochemical properties.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

The devices and methods according to the present invention have several advantages over known methods for production of chemical formulations. The use of a computer-implemented method, allow to optimize chemical recipes and formulations systematically in a Pareto Optimal way and furthermore allow for transparent decision support based on the principle of Pareto Optimality.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s).

The term “chemical formulation”, as used herein, includes, without limitation, any type of chemical formulation comprising at least two components. A component is a chemical substance composed of many identical molecules (or molecular entities) composed of atoms from more than one element held together by chemical bonds. Preferably, the chemical component is a molecule. The chemical component can also be a oligomer or polymer, or a salt formed by ionic bonds. Oligomers and polymers are composed of many similar molecules, which might differ in structure, molecular weight, composition etc. More preferably, the chemical compound is an organic molecule. The formulation may be a plasma, a gas, a liquid or a solid, may be a solution, a dispersion, an emulsion, a suspension, a sol, a gel, or a solid, or any kind of multiphase system formed by an arbitrary combination of the above listed forms of matter. Preferably, the formulation is a solution, an emulsion, or a suspension, more preferably is a solution. Preferably, the formulation comprises auxiliaries and additives, in particular buffer compounds, salts, stabilizers, solvents, and the like. Also preferably, the chemical formulation is a chemical solution, preferably an aqueous solution, more preferably a buffered solution. Thus, preferably, the chemical substance formulation further comprises water. More preferably the chemical formulations is a coating formulation.

Typically, coating formulations comprise polymers, film-forming auxiliaries, pigments, fillers, dispersing aids, thickeners, buffer substances, biocides etc..

According to an embodiment of the present invention, the chemical formulation comprises one or more of: paint formulation, agricultural multi-component formulation, pharmaceutical multi-component formulation, nutrition multi-component formulation, ink multi-component formulation, chemical formulation for construction purposes, and chemical formulation used inside oil production.

The term “key physicochemical properties”, as used herein, includes, without limitation, hydrophilicity and/or lipophilicity (e.g., distribution coefficient); melting point; permeability across biological or artificial lipid membranes; solubility in water, solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactability; hygroscopicity; water content (e.g., loss on drying); concentration of impurities; hardness, chemical resistance, color stain resistance, glass transition temperature; other chemical, physicochemical and/or physical properties; and information on compatibility and stability.

The term “experimental data”, as used herein, includes, without limitation, type and amount of formulation components and process parameters such as temperature, pressure, reaction time, reactor design etc..

The term “chemical production facility”, as used herein, preferably means chemical plant.

Experimental data may origin from
Computational data
Data computed from empirical models
Data computed from physical/chemical/scientific models
Simulations on biological data
Data from environmental fate modeling
Simulation data
Tabulated data
Literature data
Estimated data
or Algorithmically augmented data

More specifically, the simulation data may origin from
Quantum Chemical computations
Molecular Dynamics simulations
coarsed grained simulations like dissipative particle dynamics
reactor flow simulation
computational fluid mechanics
thermodynamic computations
finite element simulations
solid state continuum mechanics simulations

Experimental data might be obtained by applying Design of Experiments (DOE) techniques. DOE is a statistical framework that can be used for the design and analysis of comparative experiments. DOE methods allow a researcher to create an optimal experiment based on the number of factors, the type of the underlying model, and the goals of the experiment. Experiments can be designed to gather information with the fewest possible number of “runs to obtain the desired level of data. The two main classical types of experimental designs are screening designs and response surface methodology. A screening design is one in which relatively few experimental runs are used to efficiently study a large number of experimental factors to screen out those few that are most active from the remainder that are relatively inactive over the ranges being considered. Response Surface Methodology (RSM) is an experimental technique to find the optimal response with the specified ranges of the factors. RSM designs assist in quantifying the relationships between one or more measured responses and the vital input facts. Furthermore, there are optimized designs such as the D-, A-, E-, G-Optimal Designs. These design techniques are more flexible as the above mentioned classical ones since they e.g. allow to define constrains to encode prior knowledge about restrictions and a priori known mutual dependencies between the input variables. The prior knowledge may be based on experience and/or scientific knowledge. If, for instance, it is a priori known that the glass transition temperature of a polymer must be within specified limits, and if Tg can be calculated with sufficient accuracy by the well-known Fox equation, then this Fox equation can be used to define constrains to a priori rule out all experiments from the design with infeasible Tg. Optimized designs may also be used to efficiently augment incomplete data sets. Preferentially, D-Optimal Designs are used. More preferentially, D-Optimal designs with constrains based on prior knowledge are used. Even more preferentially, these constrained D-Optimal designs are used to generate data to be able to calibrate second-order polynomial models and/or linear models.

All design techniques described above have in common that the mathematical form of the model function is postulated a priori, and after postulating the model a minimum number of experiments (or simulation runs) must be carried out to be able to calibrate the model. An entirely different approach is to subsequently design every single experiment (or simulation run) based on the prior information given by all previous ones. In this kind of approaches, it is not necessary to a priori postulate the mathematical form of the model. Preferentially, a kernel model, more perferentially a Gaussian Process type of model is build stepwise by adding experiments to those regions in the input space where the model uncertainty is the highest Preferentially, procedures as or similar to the one described in Chemie Ingenieur Technik, Volume 91, Issue 3, Pages 277-284 are used to stepwise build a pareto frontier.

Examples of the user-defined TPP (target product profile) for chemical formulations may include, but not limited to, amount and/or concentration of the components; size, volume and/or weight of the formulation; mechanical and/or rheological properties of the formulation; release profile of components; other application-relevant parameters; compatibility and stability; and other manufacturing-relevant properties.

Particular examples of the user-defined TPP for coating formulations may include, but not limited to, scratch resistance, gloss, gel time, cure time, compatibility and stability, rheological behaviour and/or viscosity, hydrophilicity and/or lipophilicity, hardness, flowability, chemical resistance.

Further examples of the user-defined TPP for chemical formulations may include, but not limited to melting point; permeability across biological or artificial lipid membranes; solubility in water, solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactability; hygroscopicity; water content (e.g., loss on drying); concentration of impurities; hardness, chemical resistance, color stain resistance, glass transition temperature; and/or other any other chemical, physicochemical and/or physical properties. In some examples, if the chemical formulation is diluted, the physicochemical properties of the chemical formulation may refer to the physicochemical characteristics of the complete formulation (e.g. herbicide formulation plus adjuvant).

The term “input data” as used herein is a broad term and is to be given its ordinary and customary meaning to a person of ordinary skill in the art and is not to be limited to a special or customized meaning. The term preferably refers to at least experimental data as specified herein above.

The term “input unit”, as used herein, includes without limitation any item or element forming a boundary configured for transferring information. In particular, the input unit may be configured for transferring information onto a computational device, e.g. onto a computer, such as to receive information. The input unit preferably is a separate unit configured for receiving or transferring information onto a computational device, e.g. one or more of: an interface, specifically a web interface and/or a data interface; a keyboard; a terminal; a touchscreen, or any other input device deemed appropriate by the skilled person. More preferably, the input unit comprises or is a data interface configured for transferring or exchanging information as specified herein below.

The term “output unit”, as used herein, includes without limitation any item or element forming a boundary configured for transferring information. In particular, the output unit may be configured for transferring information from a computational device, e.g. a computer, such as to send or output information, e.g. onto another device, e.g. a control unit, that controls and/or monitor the production process of the chemical formulation. The output unit preferably is a separate unit configured for outputting or transferring information from a computational device, e.g. one or more of: an interface, specifically a web interface and/or a data interface; a screen, a printer, or a touchscreen, or any other output device deemed appropriate by the skilled person. More preferably, the output unit comprises or is a data interface configured for transferring or exchanging information as specified herein below.

Preferably, the input unit and the output unit are configured as at least one or at least two separate data interface(s); i.e. preferably, provide a data transfer connection, e.g. a wireless transfer, an internet transfer, Bluetooth, NFC, inductive coupling or the like. As an example, the data transfer connection may be or may comprise at least one port comprising one or more of a network or internet port, a USB-port and a disk drive. The input unit and/or the output unit may also be may be at least one web interface.

The term “processing unit” as used herein is a broad term and is to be given its ordinary and customary meaning to a person of ordinary skill in the art and is not to be limited to a special or customized meaning. The term specifically may refer, without limitation, to an arbitrary logic circuitry configured for performing operations of a computer or system, and/or, generally, to a device or unit thereof which is configured for performing calculations or logic operations. The processing unit may comprise at least one processor. In particular, the processing unit may be configured for processing basic instructions that drive the computer or system. As an example, the processing unit may comprise at least one arithmetic logic unit (ALU), at least one floating-point unit (FPU), such as a math coprocessor or a numeric coprocessor, a plurality of registers and a memory, such as a cache memory. In particular, the processing unit may be a multi-core processor. The processing unit may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. The processing unit may be configured for pre-processing the input data. The pre-processing may comprise at least one filtering process for input data fulfilling at least one quality criterion. For example, the input data may be filtered to remove missing variables. Preferably, input data may be compared to at least one pre-defined threshold value, e.g. a threshold temperature, to determine whether method step (ii) is required to be performed at all. Preferably, the processing unit is configured to perform a multicriterial optimization, preferably calculation, of an optimization signal consisting of a list of input parameters leading according to the underlying model prediction to optimized target application profiles. Preferentially, the multicriterial optimization is a Pareto optimization and this list consists of pareto optimal solutions. Furthermore the optimization signal may contain a complete or approximative representation of the pareto frontier based on the underlying model function.

Pareto optimality refers herein to the concept that a solution is a Pareto improvement if a change to a different solution makes at least one objective better off without making any other objectives worse off. A Pareto improvement is Pareto optimal or Pareto efficient if no further Pareto improvement can be made. With a Pareto frontier, by restricting attention to a set of objectives that are Pareto optimal, the chemist can make trade-offs within such a set, rather than considering the full range of every parameter.

A Pareto frontier is a set of solutions in an N-dimensional objective space that are Pareto optimal in light of a defined method of evaluation of those solutions. For the purposes of forming optimization signals, an N-dimensional Pareto frontier comprises a collection of optimization signals which accommodate the objectives of optimization.

Methods for optimization in chemical process or formulations based on experimental data are in principle known in the art.

Preferentially, the approach described in Chapter 2.3 and Apendix A in [Bortz et al., Computers and Chemical Engineering 60 (2014) 354-363] is used.

In practice, model based prediction is often carried out by models including a dimension reduction algorithm. Examples are principal component regression or partial least squares models. In these models, so-called latent variables are formed and the target variables, which later serve as optimization objectives are modelled as functions of these latent variables rather than as functions of the original input variables, such as chemical composition or process parameters. If the dimension of the space defined by the latent variables is smaller than the dimension of the space defined by the original input parameter for the considered system, then the model exhibits the feature of dimension reduction, and we call the model degenerated. This degeneracy has technical consequences. If a real-world-system is described by a degenerated model with sufficiently high accuracy, then it is possible to systematically change the original input parameters, such as chemical recipe or process, in such a way, that none of the target variables of interest are significantly changed.

The space defined by the set of all possible accessible points in input space with this property is referred to as the “invariant subspace”. In the proposed approach, statistical optimality principles on the invariant subspace are combined with applied constrains on the original input parameter space to obtain a set of input parameters, e.g. chemical recipes and/or process conditions, for a chemical formulation design. The input parameters may also be referred to as design parameters and are determined in such a way, that the obtained set of input parameters exhibit optimal statistical variability and simultaneously the target variables of interest (i.e. optimization objective parameters, optimization signal) exhibit theoretically zero or in practice technically only minimal variability. The results generated by the proposed approach can be used to cover not only one or just a few lead-recipes, but the entire class of recipes covered by the invariant subspace.

The optimization signal can be a finite set of points which approximate the Pareto set within a certain accuracy. To support the decision process, it is preferred to enable a real-time navigation on the continuous Pareto set via a GUI. Therefore, a linear interpolation between the points on the Pareto set can be carried out. This interpolation is not only done in the objective space but also in the design space.

Preferably the GUI allows the user to navigate the interpolated solutions by moving sliders corresponding to the objectives.

One way the objective sliders work is illustrated in FIG. 2 for the simple case of two objectives. During slider change on one objective, a linear problem is solved such that the remaining objectives are all changed in a definite manner. In this way, the complete Pareto set is navigable and it is possible to explore visually the trade-offs between the different objectives. This visual exploration can be done in a twofold manner: on the one hand moving one slider causes the displacement of the other sliders according to the shape of the Pareto set. This interaction between the values on the sliders is able to show the best compromises between the conflicting objectives. On the other hand, the user can restrict the range of the sliders. Restricting the range of one of the objectives, will in general also affect the range of the other objectives. This restriction of the decision space is also visualized, yielding information on which alternatives remain feasible and which are now infeasible.

The definitions made above apply mutatis mutandis to the following. Additional definitions and explanations made further below also apply for all embodiments described in this specification mutatis mutandis.

The present invention further relates to an apparatus for providing assistance for producing a chemical formulation in a chemical production facility, preferably for guiding the production of a chemical formulation in a chemical production facility, comprising:
an input unit (10); an output unit (30) and
a processing unit (20) configured to:
a) receive a user input, via the input unit, wherein the user input defines:
at least one set of experimental data comprising formulation data and/or process data and/or key physicochemical properties of the ingredients and/or the formulation and a target product profile, TPP, comprising a minimum product requirement
b) perform, via the processing unit (20), multicriterial optimization based on a computational model using experimental data
c) provide, via the output unit, optimization results.

The term “apparatus”, as used herein, relates to a system of means comprising at least the aforementioned means operatively linked to each other as to allow the determination. Typical input and output units and means for carrying out the determination, in particular processing units, are disclosed above in connection with the methods of the invention. How to link the means in an operating manner will depend on the type of means included into the device. The person skilled in the art will realize how to link the means without further ado. Preferably, the means are comprised by a single apparatus. Typical apparatuses are those which can be applied without the particular knowledge of a specialized technician, in particular hand-held devices comprising an executable code, in particular an application, performing the determinations as specified elsewhere herein. The results may be given as output of raw data which need interpretation e.g. by a technician. More preferably, the output of the apparatus is, however, processed, i.e. evaluated, raw data, the interpretation of which does not require a technician. Also preferably, some functions formulation management may be performed automatically, i.e. preferably without user interaction, e.g. adjustment of a dosage of the components, or eliciting an order of a batch of components if the remaining component on stock being below a pre-determined threshold value. Further typical devices comprise the units, in particular the input unit, the processing unit, and the output unit referred to above in accordance with the method of the invention.

The input unit of the device may be configured to retrieve input data from a local storage device, e.g. a USB storage device or a sensor having stored storage segment data during storage and/or transport. The input device may, however, also receive input data from an external data storage means or directly from a sensor, e.g. via a data connection such as the internet.

The apparatus preferably is a handheld device or any type of computing device having the features as specified. The apparatus may, however, also be an apparatus configured to make use of an chemical formulation, more preferably a chemical production machine, washing machine, a dishwasher, an industrial laundry machine, a food (e.g. milk, or meat) processing machine, an animal fed processing machine, a biofuel production machine, a leather production machine, a textile production machine, a pulp and paper production machine, or a beverage production machine.

In addition to the chemical formulation management measures as specified herein above, the apparatus preferably is configured to further perform at least one of:
download relevant information including quality information, regulatory information, safety data, and/or technical documents;
order components; and/or
provide a user-feedback including usability, information content and/or chemical formulation outcome.

The present invention also relates to a system
providing assistance for producing a chemical formulation, preferably for guiding the production of a chemical formulation, comprising:
   an apparatus according to claim 10; and
   a web server configured to interface with a user via a webpage and/or an application program served by the web server;
wherein the apparatus is configured to provide a graphical user interface, GUI, to a user, by the webpage and/or the application program.

The term “system” as used herein is a broad term and is to be given its ordinary and customary meaning to a person of ordinary skill in the art and is not to be limited to a special or customized meaning. The term includes, without limitation, any a setup having at least two interacting components. Specifically, the term may include any type of system comprising the components as specified. Preferably, the apparatus comprised in the system is an apparatus as specified herein above. Preferably, the apparatus is a computing device comprising a data interface as an input unit and as an output unit. Thus, preferably, the apparatus comprised in the system preferably is configured to receive input data from an external data storage means or directly from a sensor, e.g. via a data connection such as the internet.

The system is configured to output a optimisation signal to an external data storage means and/or processing device, preferably a handheld device or remote computing device, via a web server configured to interface with a user via a webpage served by the web server and/or via an application program, wherein the system is configured to provide a graphical user interface (GUI) to a user by the webpage and/or the application program. Thus, preferably, the server is configured to provide a graphical user interface (GUI) to a user by the webpage and/or the application program. The term “graphical user interface” is known to the skilled person to relate to a user interface allowing a user to interact with an electronic device, in particular an apparatus or other computing device, through visual indicators instead of text-based user interaction, such as typed commands or text navigation. Also the term “application program” abbreviated as “application” or “App”, is also known to the skilled person as a computer executable code, in particular a software program providing a graphical user interface for a computing device function or a specific application of a computing device. Preferably, the application program is an executable code opening the web page served by the apparatus as specified elsewhere herein, preferably on a handheld device.

As the skilled person will understand in view of the present description, the web server may serve optimisation signal as such;

The present invention also relates to a computer program comprising instructions which, when the program is executed by the apparatus of the present invention, specifically by a processor of the apparatus, and/or by the system of the present invention, cause the apparatus and/or the system to perform the method of the present invention.

The present invention also relates to a computer-readable storage medium comprising instructions which, when executed by the apparatus of any one of the present invention and/or the system of any one of the present invention, cause the apparatus and/or the system to perform the method of the present invention.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

The present invention also relates to a method for monitoring the production process of a chemical formulation, the method comprising the steps of:
providing (220) a target performance characteristic of a chemical formulation;
providing (222) a performance characteristic of a produced chemical formulation generated according to the method of any one of the preceding claims; and
comparing (224) the performance characteristic with the target performance characteristic of the chemical formulation to determine if the produced chemical formulation fulfils predetermined quality criteria.

A comparison between the measured performance characteristics and the target performance characteristics of the chemical formulation allows not only for quality control or more reliable production but may be extended via a feedback loop which adjusts the production process, where needed.

This will be explained in detail hereinbelow and in particular with respect to the embodiments shown in FIGS. 4 and 6.

The present invention also relates to a method for validating the production of a chemical formulation, the method comprising the steps of:
providing (234) an existing performance characteristic for a chemical formulation that has been produced from validated precursors;
generating (236) a optimization signal based on the existing performance characteristic according to the method of any one of claims 1 to 9, wherein the optimization signal comprises an ingredient identifier and related property data, which are associated with at least one new precursor; and
comparing a performance characteristic of a chemical formulation produced using the optimization signal and the existing performance characteristic to validate the at least one new precursor.

This will be explained in detail hereinbelow and in particular with respect to the embodiments shown in FIGS. 5 and 7.

The present invention also relates to an apparatus for monitoring production of a chemical formulation, the apparatus comprising one or more processing unit(s) configured to monitor production, wherein the processing unit(s) include instructions, which when executed on the one or more processing unit(s) execute the following method steps:
providing (220) a target performance characteristic of a chemical formulation;
providing (222) a performance characteristic of a produced chemical formulation generated according to the method of any one of claims 1 to 9; and
comparing (224) the performance characteristic with the target performance characteristics of the chemical formulation to determine if the produced chemical formulation fulfils predetermined quality criteria.

The present invention also relates to an apparatus for validating production of a chemical formulation, the apparatus comprising one or more processing unit(s) configured to validate production, wherein the processing unit(s) include instructions, which when executed on the one or more processing unit(s) execute the following method steps:
providing (234) an existing performance characteristic for a chemical formulation that has been produced from validated precursors;
generating (236) an optimization signal based on the existing performance characteristic according to the method of any one of claims 1 to 6, wherein the optimization signal comprises an ingredient identifier and related property data, which are associated with at least one new precursor; and
comparing a performance characteristic of a chemical formulation produced using the optimization signal and the existing performance characteristic to validate the at least one new precursor.

### FIGURE LEGENDS

FIG. 1: Device/system

FIG. 2: Pareto Sliders

FIG. 3: interface for user interaction

FIG. 4 shows an example of a flowchart for monitoring quality of the chemical formulation in a production process of the chemical formulation having a target performance characteristics.

FIG. 5 shows an example of a flowchart for validating the production of the chemical formulation.

FIG. 6 shows an example of a production line for producing the chemical formulation with a monitoring apparatus.

FIG. 7 shows another example of a production line for producing the chemical formulation with a validation apparatus.

The following Examples shall merely illustrate the invention. They shall not be construed, whatsoever, to limit the scope of the invention.

### EXAMPLE 1

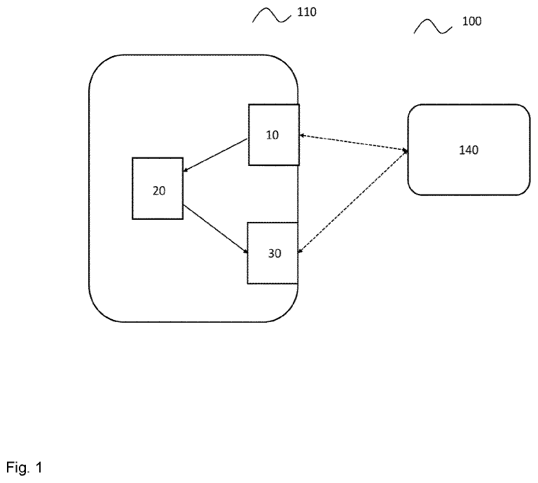

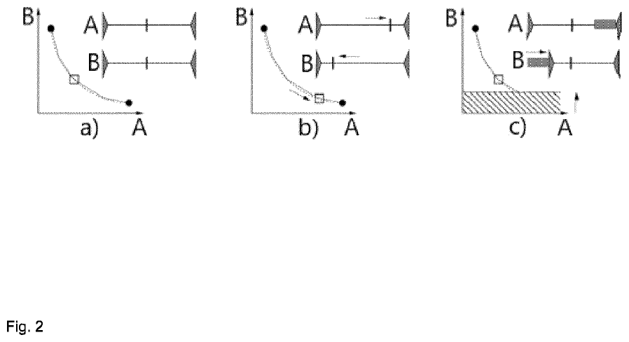

As shown in FIG. 1, a system 100 for providing assistance for producing a chemical formulation in a chemical production facility, preferably for guiding the production of a chemical formulation in a chemical production facility is disclosed. The system 100 comprises an apparatus 110 for formulation management of an chemical formulation and, further, a web server 140 configured to interface with a user via a webpage served by the web server and/or via an application program. The apparatus 110 comprises an input unit 10, a processing unit 20, and an output unit 30. In the system 100, the web server 140 may communicate with the input unit 10 and/or the output unit 30.

Apparatus 110 comprises at least one processing unit 20 such as a processor, microprocessor, or computer system, in particular for executing a logic in a given algorithm. The apparatus 110 may be configured for performing and/or executing at least one computer program of the present description. The processing unit 30 may comprise at least one processor. In particular, the processing unit 30 may be configured for processing basic instructions that drive the computer or system. As an example, the processing unit 30 may comprise at least one arithmetic logic unit (ALU), at least one floating-point unit (FPU), such as a math co-processor or a numeric coprocessor, a plurality of registers and a memory, such as a cache memory. In particular, the processing unit 30 may be a multi-core processor. The processing unit 30 may be configured for machine learning. The processing unit 30 may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like.

The apparatus comprises at least one communication interface, preferably an output unit 30, configured for outputting data. The communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information, i.e. may be an input unit 10. The communication interface may specifically provide means for transferring or exchanging information. In particular, the communication interface may provide a data transfer connection, e.g. Blue-tooth, NFC, inductive coupling or the like. As an example, the communication interface may be or may comprise at least one port comprising one or more of a network or internet port, a USB-port and a disk drive. The communication interface may be at least one web interface. The input data comprises storage segment data as specified herein above.

The processing unit 20 may be configured for pre-processing the input data. The pre-processing unit 20 may comprise at least one filtering process for input data fulfilling at least one quality criterion. The processing unit 20 is configured for determining at least one optimization signal, preferably as specified herein above and below in the further Examples.

The web server 140 is configured to provide a GUI for the apparatus 110. Thus, the web server may exchange data with the output unit 30, e.g. for displaying said data on the GUI. The web server 140 may, however, also exchange data with the input unit of the apparatus.

### EXAMPLE 2

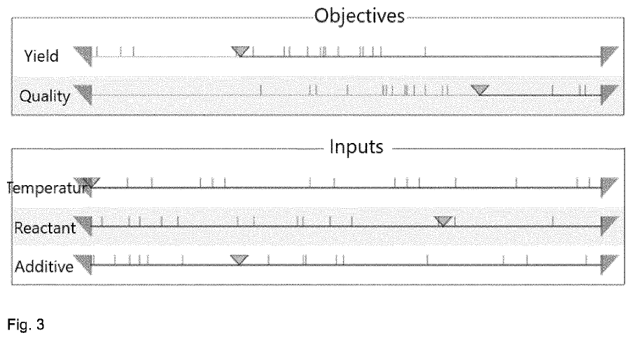

A web interface for user interaction may be configured as exemplarily shown in FIG. 3. FIG. 3 shows a possible implementation of the user interface in form of interactively movable sliders. In this example three inputs and two objectives are considered. By moving one of the objective sliders the other one is automatically adjusted by the system to follow the pareto frontier. At the same time, the corresponding input parameter combination leading to the corresponding position on the Pareto Frontier is computed and indicated by an automatic positioning of the input sliders.

Reference signs:
10 input unit
20 processing unit
30 output unit
100 system
110 apparatus
140 web server

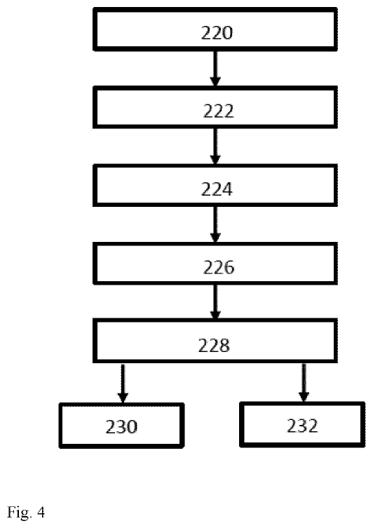

FIG. 4 shows an example of a flowchart for monitoring quality of the chemical formulation in a production process of the chemical formulation having target performance characteristic of the chemical formulation.

In step 220, the target performance characteristics are provided e.g. from a user input.

In step 222, the performance characteristic of the produced chemical formulation is provided. The produced chemical formulation is generated according to the method described therein to meet the target performance characteristic.

The performance characteristic may be provided by or derived from measurement data. Such measurement data for instance includes measurement data provided by one or more sensors, such as optical sensors. The one or more sensors may be used to measure the physicochemical properties of the produced chemical formulation. For drug formulation, the measured physicochemical properties could include one or more of the following parameters: solvents, co-solvents and/or biorelevant media; miscibility with water, solvents, co-solvents and/or biorelevant media; true density; viscosity; wettability; interfacial and/or surface tension; particle size distribution data; particle morphology, shape and/or aspect ratio; bulk and tapped density; flowability (e.g., angle of repose or flow function coefficient); compressibility and compactability; hygroscopicity; water content (e.g., loss on drying); concentration of impurities; hardness, chemical resistance, color stain resistance, glass transition temperature; and/or other any other chemical, physicochemical and/or physical properties.

In step 224, the performance characteristic as provided or measured may be compared to the target performance characteristic of the chemical formulation to determine if the produced chemical formulation fulfils predetermined quality criteria.

The comparison may performed by comparing one or more physical, chemical or physio-chemical characteristic(s) that relate to the performance characteristic.

Optionally, in step 226, the target performance characteristics may be mapped to the performance characteristics. In other word the values corresponding to the performance characteristics may be determined from target performance characteristics. In other embodiments the performance characteristic may be mapped to the target performance characteristics. Both options are equally applicable.

Optionally, in step 228, the target performance characteristics and the performance characteristics or any corresponding values derived therefrom are used for validation. Such validation may be performed by comparing values or value ranges.

If the values lie within an acceptable range or value, such as a 1- or 2-standard deviation(s) interval, the chemical formulation as measured may be valid in the sense that it fulfils the performance criterium or criteria. If the values do not lie within an acceptable range, such as a 1- or 2-standard deviation(s) interval, the chemical formulation as measured may be invalid in the sense that it does not fulfil the performance criterium or criteria.

Optionally, if the chemical formulation is valid, e.g. a control signal for a production process may be triggered in step 230. Such control signal may be associated with the composition of the chemical formulation. It may control dosing equipment for dosing of different components of the chemical formulation in the production process.

Optionally, if the chemical formulation is invalid, e.g. a warning signal for the operator of the production process may be triggered in step 232. Such warning signal may signify the invalidity of the chemical formulation. The invalidity may trigger a stop signal for the production process. In such cases, the optimization signal may be updated for the production of the chemical formulation to achieve the target performance characteristics of the chemical formulation.

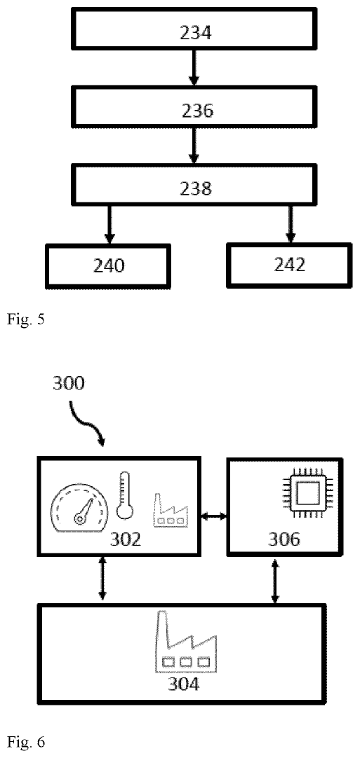

FIG. 5 shows an example of a flowchart for validating the production of the chemical formulation.

In step 234, an existing performance characteristic (e.g. one or more measured physicochemical properties) for a chemical formulation is provided, which has been produced from validated precursors.

In step 236, based on the existing performance characteristic a optimization signal is generated according to the method described therein that includes an ingredient identifier and related property data, which are associated with at least one new precursor.

In step 238, the performance characteristic of a chemical formulation produced based on the optimization signal and the existing performance characteristics are compared to validate the at least one new precursor. If the comparison lies within an acceptable range, the at least one new precursor is valid. On the other hand, if the comparison does not lie within the acceptable range, the at least one new precursor is invalid.

If new precursor(s) is valid, e.g. control signal is generated for a production process based on the new precursor(s) may be triggered in step 240. Such control signal may by be associated with the composition of the chemical formulation including the new precursor. It may control dosing equipment configured to dose different components of the chemical formulation in the production process.

If the chemical formulation is invalid, e.g. a warning signal for the operator of the production process may be triggered in step 242. Such warning signal may signify the invalidity of the new precursor(s). This may trigger a stop signal for the production process.

FIG. 6 shows an example of a production line 300 for producing the chemical formulation with a monitoring apparatus 306.

The production line 300 may include dosing equipment 302 configured to dose different precursors of the chemical formulation in the production process. The production line may include a conveyor system 304 to convey e.g. bottles, plastic packaging or other suitable packaging to be filled with the chemical formulation. The production line may include a monitoring apparatus 306 configured to monitor quality of the chemical formulation in a production process of the chemical formulation.

The monitoring apparatus 306 and/or the dosing equipment apparatus 302 may be configured to receive a target performance characteristics of the chemical formulation. The target performance characteristics may specify the composition data for the chemical formulation including one or more ingredients. The target performance characteristics may include quality criteria like physiochemical properties. The monitoring apparatus may be configured to provide the composition data to the dosing equipment and vice versa. The dosing equipment may be configured to control the dosing based on the provided composition data.

The monitoring apparatus 306 may be configured to measure one or more performance characteristic(s). The monitoring apparatus 306 may be configured to compare the physiochemical properties, or any value derived from the physiochemical properties to the measured performance characteristic(s). If the comparison lies within an acceptable range or value, the produced chemical formulation fulfills quality criteria. If the comparison does not lie within an acceptable range or value, the produced chemical formulation does not fulfill quality criteria. In the latter case the monitoring unit may be configured to notify an operator or to provide adjusted composition data to the dosing equipment 302.

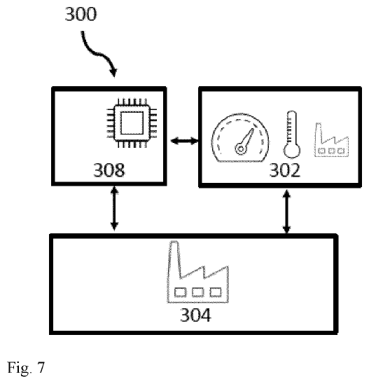

FIG. 7 shows another example of a production line 300 for producing the chemical formulation with a validation apparatus 308.

The production line 300 may include dosing equipment 302 configured to dose different precursors of the chemical formulation in the production process. The production line 300 may include a conveyor system 304 to convey e.g. bottles, plastic packaging or other suitable packaging to be filled with the chemical formulation. The production line 300 may include a validation apparatus 308 configured to validate the production of the chemical formulation.

The validation apparatus 308 may be configured to receive an existing performance characteristic of the chemical formulation (e.g. two or more physicochemical properties or any value derived from the physicochemical properties). The validation apparatus 308 may be configured to generate a optimization signal based on the existing performance characteristic. The optimization signal may comprise new precursor(s). The validation apparatus 308 may be configured to receive one or more data associated with the new precursor(s). The validation apparatus 308 may be configured to validate the new precursor(s) for production of the chemical formulation. The validation apparatus 308 may be configured to compare a performance characteristic of a chemical formulation produced using the new optimization signal and the existing performance characteristic. This way not only the production of the chemical formulation but also its application may be validated. The validation apparatus 308 may be configured to provide the composition data including the new precursor(s) to the dosing equipment and vice versa.

Combinations and modifications of the embodiments shown in FIGS. 4 and 5 are similarly possible. Both methods exemplify the strength of the methods described herein. This allows for simplified and more reliable production through monitoring production of the chemical formulation or through validating new precursor(s) to be used for producing the chemical formulation.

1. A computer implemented method for producing a chemical formulation in a chemical production facility, preferably for guiding the production of a chemical formulation, comprising:
(a) receiving input data, preferably via an input unit, of at least one set of experimental data comprising formulation data and/or process data and/or key physicochemical properties of the components and/or the formulation and a target product profile, TPP, comprising a minimum product requirement
(b) performing multicriterial optimization based on a computational model based on experimental data via a processing unit);
(c) providing optimization signal, preferably via an output unit,
wherein the optimization signal is configured to control and/or monitor, preferably via a control unit, the production process of the chemical formulation.

2. The method according to claim 1, wherein the multi-criteria optimization is based on the set of experimental data, to construct a Pareto frontier, wherein the set of experimental data are evaluated with at least two objectives measuring qualities of the set of experimental data, wherein formulations on the constructed Pareto frontier are Pareto optimal with respect to the objectives.

3. The method according to claim 2, wherein the optimization results are provided in a way that navigation on the pareto frontiers is possible.

4. The method according to claim 1, wherein the optimization results are provided in a way that the entire class of results covered by the invariant subspace is accessible.

5. The method according to claim 1, wherein the input data is generated via Design of Experiments (DoE) technique.

6. The method according to claim 1, wherein the formulation data comprises formulation components and amounts of formulation components.

7. The method according to claim 5, wherein the Design of experiments (DoE) is based on a Gaussian Process model.

8. The method according to claim 5, wherein the Design of experiments (DoE) is based on a kernel model.

9. The method according to claim 1, wherein the chemical production facility is a chemical plant.

10. An apparatus for producing a chemical formulation in a chemical production facility, preferably for guiding the production of a chemical formulation, comprising:
an input unit; an output unit and
a processing unit configured to:
a) receive a user input, via the input unit, wherein the user input defines:
at least one set of experimental data comprising formulation data and/or process data and/or key physicochemical properties of the ingredients and/or the formulation and a target product profile, TPP, comprising a minimum product requirement
b) perform, via the processing unit, multicriterial optimization based on a computational model using experimental data
c) provide, via the output unit, optimization results.

11. A system for providing assistance for optimizing chemical formulations, comprising:
an apparatus according to claim 10; and
a web server configured to interface with a user via a webpage and/or an application program served by the web server;
wherein the apparatus is configured to provide a graphical user interface, GUI, to a user, by the webpage and/or the application program.

12. A computer program element comprising sets of instructions,
wherein, when the sets of instructions are executed on a processor of the apparatus of claim 11, the sets of instructions cause the apparatus or the system to perform the method of claim 1.

13. A method for monitoring the production process of a chemical formulation, the method comprising:
providing a target performance characteristic of a chemical formulation;
providing a performance characteristic of a produced chemical formulation generated according to the method of claim 1; and
comparing the performance characteristic with the target performance characteristics of the chemical formulation to determine if the produced chemical formulation fulfils predetermined quality criteria.

14. A method for validating the production of a chemical formulation, the method comprising:
providing an existing performance characteristic for a chemical formulation that has been produced from validated precursors;
generating a optimization signal based on the existing performance characteristic according to the method of claim 1, wherein the optimization signal comprises an ingredient identifier and related property data, which are associated with at least one new precursor; and
comparing a performance characteristic of a chemical formulation produced using the optimization signal and the existing performance characteristic to validate the at least one new precursor.

15. An apparatus for monitoring production of a chemical formulation, the apparatus comprising one or more processing unit(s) configured to monitor production, wherein the processing unit(s) include instructions, which when executed on the one or more processing unit(s) execute the method steps of claim 13.

16. An apparatus for validating production of a chemical formulation, the apparatus comprising one or more processing unit(s) configured to validate production, wherein the processing unit(s) include instructions, which when executed on the one or more processing unit(s) execute the method steps of claim 14.

17. (canceled)
